(19) United States
(12) Patent Application Publication
(10) Pub. No.: US 2026/0021249 A1
(43) Pub. Date: Jan. 22, 2026

(54) SEMI-AUTOMATED, DYNAMIC SYSTEM WITH MECHANICAL FEEDBACK FOR INTRA-MYOCARDIAL INJECTION

(71) Applicant: UNIVERSITY OF SOUTH CAROLINA, COLUMBIA, SC (US)

(72) Inventors: AHMED ALSHAREEF, COLUMBIA, SC (US); TAREK SHAZLY, COLUMBIA, SC (US); FRANCIS SPINALE, BLYTHEWOOD, SC (US); AMRO ALSHAREEF, COLUMBIA, SC (US)

(21) Appl. No.: 19/203,259

(22) Filed: May 9, 2025

Related U.S. Application Data
(60) Provisional application No. 63/673,236, filed on Jul. 19, 2024.

(51) Int. Cl. A61M 5/172; A61M 5/145; A61M 5/158

(52) U.S. Cl. CPC A61M 5/1723; A61M 5/14546; A61M 5/158; A61M 2005/1583; A61M 2005/1585; A61M 2005/1586; A61M 2005/1588; A61M 2005/1726; A61M 2210/125; A61M 2230/06; A61M 2230/63

(57) ABSTRACT

The disclosure deals with a semi-automated, dynamic system and corresponding method using mechanical feedback for intra-myocardial injections. A minimally invasive injection device enters through the anterior chest cavity to deliver a therapeutic payload to the intra-myocardium. The device includes, in part, a needle paired with a mechanical feedback system of translatable material which is able to sense linear motion. The system moves back and forth against the material when in contact with the heart, with a displacement sensor measuring the amount of movement. A syringe injection motor pushes the syringe for automated injection, controlled by a microcontroller operating per a control algorithm. The control algorithm uses the mechanical feedback system to automatically sense the temporal response of the heartbeat to map deformation activity of the heart in the region around injection. A closed-loop system automatically guides injection.

### PRIORITY CLAIM

The present application claims the benefit of priority of U.S. Provisional Patent Application No. 63/673,236, filed Jul. 19, 2024, which is titled Semi-Automated, Dynamic System With Mechanical Feedback For Intra-Myocardial Injection, and which is fully incorporated herein by reference for all purposes.

### STATEMENT REGARDING FEDERALLY SPONSORED RESEARCH OR DEVELOPMENT

This invention was made with government support under R01 HL167994 awarded by the National Institutes of Health. The government has certain rights in the invention.

### BACKGROUND OF THE PRESENTLY DISCLOSED SUBJECT MATTER

The disclosure deals with a semi-automated, dynamic system and corresponding method using mechanical feedback for intra-myocardial injections.

Approximately, 550,000 first episodes and 200,000 recurrent episodes (750,000 combined episodes) of acute myocardial infarction occur annually. Intramyocardial injection therapy is performed by injection into the diseased (targeted) section of the heart wall.

FIG. 1 represents a diagram of a Prior Art targeted intramyocardial injection methodology using a semi-automated delivery platform to deliver therapeutic payloads (for example such as injectates) to the infarct heart directly (no open chest surgery), as shown and discussed in International Patent Application Number PCT/US2021/050184. The complete disclosure of International Patent Application Number PCT/US2021/050184 is fully incorporated herein for all purposes. Medical device 10 is intended for performing controlled intramyocardial injections by utilizing the transepicardial route of direct administration, and includes a multipurpose needle 12, control systems 14, delivery apparatus 16, and a therapeutic agent 18. An intrusion needle is guided through the chest wall and positioned near the beating heart of the study/treatment subject, with placement supported by ultrasonic visualization of the heart in the chest cavity, fluoroscopy, or other real-time cardiac mapping techniques. After insertion of the intrusion needle (primary needle), a probing device 20 can be inserted through the primary needle to detect/locate the site of injection on the myocardium. Probing device or module 20 can be a fiber angioscope that produces real-time images that can be analyzed by the operator or the controller software, or it may be a conductive probe that measures bio-impedance of the myocardium. Control software may include software used to “drive or control” the motors and the pumping of medication in an infusion pump. The software may also encrypt data for transmission from the probing device 20.

After identification of the site of injection, a secondary needle 22 (smaller than the primary needle), may be guided through the intruding or primary needle). The primary needle may contact the region of interest, with injection made only when such contact is made.

The injection control is based on the real-time electrocardiogram (ECG) of the patient or study subject. Injection commands can be sequenced and processed to ensure automated injections during the diastolic period. Injectable biomaterial/bioactive agents (i.e., the payload) can be infused via a hydrodynamic system that uses a precision pump 24 in order to control the payload delivery.

The presently disclosed subject matter relates to a minimally invasive injection device that enters through the chest cavity (for example, anterior) to deliver a therapeutic payload to the intra-myocardium. Therefore, the presently disclosed medical device technology has the potential to reduce costs, reduce patient recovery times, reduce error associated with manual drug delivery, reduce risk of complications with open heart surgery, while giving surgeons more information about heart function using a minimally invasive device.

### SUMMARY OF THE PRESENTLY DISCLOSED SUBJECT MATTER

The presently disclosed subject matter deals with semi-automated, dynamic systems and corresponding methodologies using mechanical feedback for intra-myocardial injections.

For some presently disclosed embodiments, for example, a minimally invasive injection device enters through the anterior chest cavity to deliver a therapeutic payload to the intra-myocardium. For some such embodiments, a device may include, in part, a needle paired with a mechanical feedback system of translatable material which is able to sense linear motion. The system may move back and forth against the material when in contact with the heart, with a displacement sensor measuring the amount of movement. A syringe injection motor may push the syringe for automated injection, controlled by a microcontroller operating per a control algorithm. The control algorithm may use the mechanical feedback system to automatically sense the temporal response of the heartbeat to map deformation activity of the heart in the region around injection. A closed-loop system may automatically guide injection for some embodiments.

In one exemplary embodiment disclosed herewith, system and corresponding and/or associated method are provided for semi-automated delivery of therapeutic payloads to a heart.

It is to be understood that the presently disclosed subject matter equally relates to associated and/or corresponding methodologies. One exemplary such method relates to a method for semi-automated delivery of therapeutic payloads to a heart, comprising introducing through a patient's opened chest cavity an injection device supporting a needle and a movable physical sensor probe; positioning the movable physical sensor probe into contact with the exposed heart of the patient; tracking the beat pattern of the patient's heart by maintaining contact of the movable physical sensor probe with the patient's heart through its beat cycle; and inserting the needle into the patient's heart and injecting a therapeutic payload via the needle.

Another exemplary such method relates to a method for performing automated, targeted payload injection into the intra-myocardium of a patient, comprising controllably introducing a minimally invasive injection device through the patient's opened chest cavity; and sensing the mechanical and electrical signals of the patient's heart and dynamically injecting a known quantity of therapeutic payload within a precise time window at a specific location, via a needle carried by the injection device.

Other example aspects of the present disclosure are directed to systems, apparatus, tangible, non-transitory computer-readable media, user interfaces, memory devices, and electronic devices for semi-automated delivery of therapeutic payloads to a heart. To implement methodology and technology herewith, one or more processors may be provided, programmed to perform the steps and functions as called for by the presently disclosed subject matter, as will be understood by those of ordinary skill in the art.

Another exemplary embodiment of presently disclosed subject matter relates to a system for semi-automated delivery of therapeutic payloads to a heart, comprising an injection device supporting a needle and a movable physical sensor probe; a mounting component for supporting the injection device introduced through a patient's opened chest cavity, with the movable physical sensor probe into contact with the exposed heart of the patient; a displacement sensor associated with the movable physical sensor probe, for measuring the amount of movement of the patient's heart while the movable physical sensor probe maintains contact with the patient's heart through its beat cycle; an ECG device for providing electrical signals from the patient's heart; a syringe associated with the needle for delivery of a predetermined drug to a patient's heart; a syringe injection motor for controlling injection of the syringe; a needle position motor for controlling the depth of the needle into a patient's heart; and one or more processors programmed for tracking the beat pattern of the patient's heart by signals from the displacement sensor and the ECG device, and controlling the needle position motor and syringe injection motor for automatically inserting the needle into the patient's heart and injecting a therapeutic drug from the syringe via the needle.

Additional objects and advantages of the presently disclosed subject matter are set forth in, or will be apparent to, those of ordinary skill in the art from the detailed description herein. Also, it should be further appreciated that modifications and variations to the specifically illustrated, referred and discussed features, elements, and steps hereof may be practiced in various embodiments, uses, and practices of the presently disclosed subject matter without departing from the spirit and scope of the subject matter. Variations may include, but are not limited to, substitution of equivalent means, features, or steps for those illustrated, referenced, or discussed, and the functional, operational, or positional reversal of various parts, features, steps, or the like.

Still further, it is to be understood that different embodiments, as well as different presently preferred embodiments, of the presently disclosed subject matter may include various combinations or configurations of presently disclosed features, steps, or elements, or their equivalents (including combinations of features, parts, or steps or configurations thereof not expressly shown in the figures or stated in the detailed description of such figures). Additional embodiments of the presently disclosed subject matter, not necessarily expressed in the summarized section, may include and incorporate various combinations of aspects of features, components, or steps referenced in the summarized objects above, and/or other features, components, or steps as otherwise discussed in this application. Those of ordinary skill in the art will better appreciate the features and aspects of such embodiments, and others, upon review of the remainder of the specification, and will appreciate that the presently disclosed subject matter applies equally to corresponding methodologies as associated with practice of any of the present exemplary devices, and vice versa.

These and other features, aspects and advantages of various embodiments will become better understood with reference to the following description and appended claims. The accompanying drawings, which are incorporated in and constitute a part of this specification, illustrate embodiments of the present disclosure and, together with the description, serve to explain the related principles.

### BRIEF DESCRIPTION OF THE FIGURES

A full and enabling disclosure of the present subject matter, including the best mode thereof to one of ordinary skill in the art, is set forth more particularly in the remainder of the specification, including reference to the accompanying figures in which:

FIG. 1 illustrates a diagram of a Prior Art targeted intramyocardial injection methodology using a semi-automated delivery platform to deliver therapeutic payloads to the infarct heart directly (no open chest surgery);

FIGS. 2(A) and 2(B) respectively illustrate side elevation and top elevation views of 3D models of design schematics of an exemplary presently-disclosed injection device for delivering a therapeutic payload to the intra-myocardium;

FIG. 3(A) illustrates an isometric, partially side/partially top view of an image of an exemplary embodiment of the subject matter represented by FIG. 2(A), mounted on a supporting surgical arm;

FIG. 3(B) illustrates an enlarged view of a distal end of an image of an exemplary embodiment of the subject matter represented by FIG. 2(A), particularly showing a needle and physical sensor probe;

FIG. 3(C) illustrates an image of an overall perspective view of the subject matter of FIG. 3(A);

FIG. 3(D) illustrates an image of similar subject matter as that of FIG. 3(C) but from a different perspective;

FIG. 3(E) illustrates an image of an enlarged view of a portion of the FIG. 3(D) subject matter;

FIGS. 4(A) through 4(D) show successive images of an exemplary embodiment of an injection device or component of the presently disclosed subject matter engaging a plastic test material, in accordance with presently disclosed methodology, with the successive images of FIGS. 4(A) through 4(D) representing demonstration of a sequence in accordance with presently disclosed subject matter for injection of a drug (dose) through a representative plastic barrier;

FIGS. 5(A) and 5(B) respectively illustrate images of a surgical set up of the presently disclosed subject matter during use, from opposite side perspectives relative to a subject during open-heart porcine (pig) surgery; and

FIGS. 6(A) through 6(F) illustrate successive images representing demonstration of a sequence in accordance with presently disclosed subject matter for injection of a drug (dose) into a porcine subject heart.

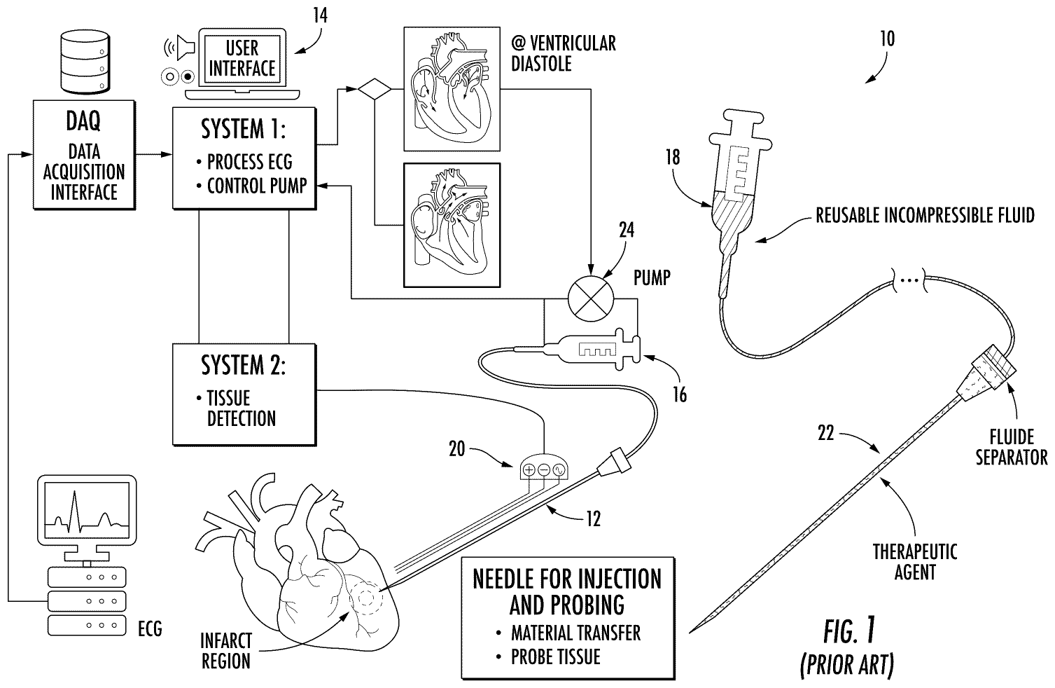

Repeat use of reference characters in the present specification and drawings is intended to represent the same or analogous features, elements, or steps of the presently disclosed subject matter. Likewise, use of reference characters in one figure is intended to represent same or corresponding features in other figures, even if such reference characters are not actually applied to the other figures.

### DETAILED DESCRIPTION OF THE PRESENTLY DISCLOSED SUBJECT MATTER

Reference will now be made in detail to various embodiments of the disclosed subject matter, one or more examples of which are set forth below. Each embodiment is provided by way of explanation of the subject matter, not limitation thereof. In fact, it will be apparent to those skilled in the art that various modifications and variations may be made in the present disclosure without departing from the scope or spirit of the subject matter. For instance, features illustrated or described as part of one embodiment, may be used in another embodiment to yield a still further embodiment.

In general, the present disclosure is directed to system and corresponding and/or associated methodology dealing with, for example, a semi-automated, dynamic technology using mechanical feedback for intra-myocardial injections. A minimally invasive injection device enters through the anterior chest cavity to deliver a therapeutic payload to the intra-myocardium. The device may include, in part, a needle paired with a mechanical feedback system of translatable material which is able to sense linear motion. The system moves back and forth against the material when in contact with the heart, with a displacement sensor measuring the amount of movement. A syringe injection motor pushes the syringe for automated injection, controlled by a microcontroller operating per a control algorithm. The control algorithm uses the mechanical feedback system to automatically sense the temporal response of the heartbeat to map deformation activity of the heart in the region around injection. A closed-loop system automatically guides injection.

Existing methods of drug delivery to injured or diseased cardiac tissue often involve invasive surgery (e.g. open heart) that can lead to complications and long recovery times. Additionally, the payload is typically delivered manually without much accuracy or precision on location or quantity. The presently disclosed subject matter allows for minimally invasive intra-myocardial drug delivery while also being able to sense the electrical and mechanical signals of the heart, allowing for automated injections and reducing user error.

For example, the presently disclosed injection device is designed to allow for automated, targeted payload injection into the intra-myocardium following injury. The physical components of the heart allow the device to sense the mechanical and electrical signals of the heart to be able to dynamically inject a known quantity of therapeutic payload within a precise time window at a specific location. Using a closed feedback system of sensors and motors, the device can semi-automatically carry out injections to the moving (i.e., on-beat) heart with minimal user control.

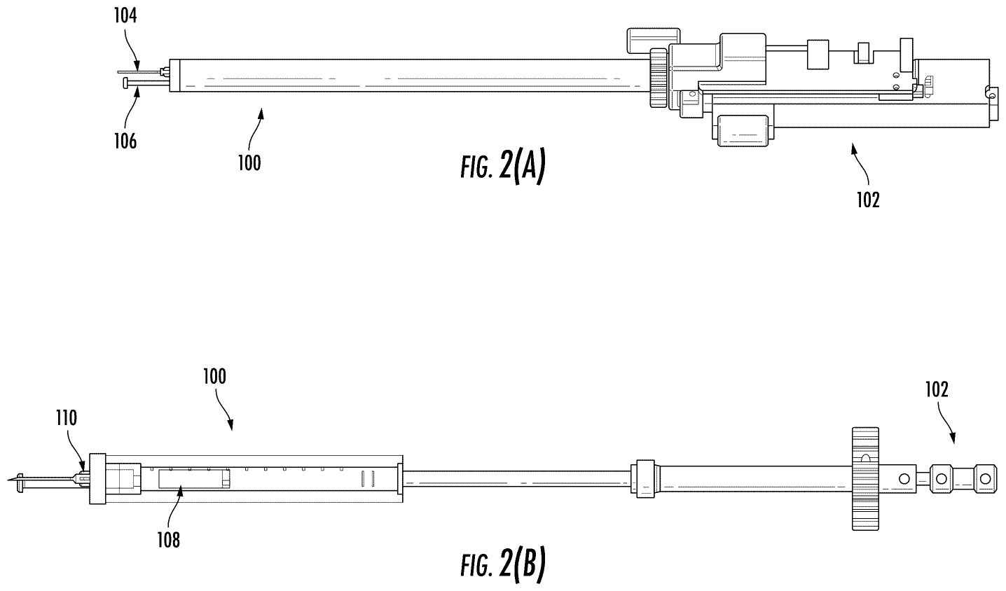

FIGS. 2(A) and 2(B) respectively illustrate side elevation and top elevation views of an exemplary presently-disclosed minimally invasive injection device or component generally 100 that enters through the chest cavity to deliver a therapeutic payload to the intra-myocardium. As shown, such exemplary embodiment is currently about 16 mm in diameter at insertion, which for example may be through the anterior chest cavity of a patient.

A number of physical components comprise aspects of the presently disclosed exemplary embodiment. For example, while the exemplary device generally 100 may be about 24 cm in length, it may be supported by a mounting component generally 102, for connecting device 100 to a surgical arm. Such component 102 may be about 15 cm long, and have both wire management features, as well as safety limit features regarding movement as well as related to a syringe feature, as discussed herein.

A needle generally 104 may be supported on the device 100. As shown (see particularly FIG. 2(B)), about a 1 cc syringe may be associated with the needle, which may be about 20 gauge or smaller, and be replaceable.

The presently disclosed subject matter includes a dynamic feedback system, which includes a translatable physical sensor probe generally 106 that is able to sense linear motion. The physical probe 106 will move back and forth against the material when in contact with the heart. A displacement sensor will measure the amount of movement of sensor probe 106 to be able to inform the sensing system and automated injection. Other presently disclosed options to sense the local tissue motion may include a conductance sensor measurement, imaging based measurement (e.g. ultrasound, optical), or mechanical impedance measurement.

Another aspect of respective FIGS. 2(A) and 2(B) is that the injection device or component generally 100 is respectively shown in retracted and at least partially extended positions thereof relative to the mounting component 102. As illustrated in FIG. 2(B), component 100 may extend from mounting component 102 via a physical supporting structure, such as a set of respectively telescoping members.

The presently disclosed subject matter may further include an electrical measurement system, including for example conductive electrodes or material to provide a secondary measurement of the mechanical feedback system, and to map local tissue dysfunction or necrosis. Such feature also provides high temporal resolution ECG measurements to guide automated injection to non-healthy tissue. Such system features may be integrated into the physical parts of dynamic feedback system or as a separate attachment or feature.

Another aspect of the exemplary presently disclosed subject matter may further include a payload cartridge generally 108, such as a plastic syringe capable of carrying a prescribed dosage volume of drug.

An additional aspect of the exemplary presently disclosed subject includes a syringe injection motor generally 110, to push the syringe with high speed and precision to eject drug into the tissue automatically. Motion 110 is preferably selected to be able to push with high force (e.g. 250 N) and at high speed (e.g.1 mL/s) for payloads that include water-based, shear thinning, or viscous materials (e.g. hydrogels). The payload to be injected per presently disclosed subject matter may, for example, comprise biomaterial, bioactive substances, cells, or combinations thereof. Motion 110 has high precision and accuracy, and is equipped with high-precision encoder features to accurately measure position and speed of the motor.

Still another aspect of the exemplary presently disclosed subject relates to use of a rotational position motor, to allow 2D (in-plane) control of the needle injection site as well as specifically positions of the needle, the dynamic feedback system, and the electrical measurement system. Such motor needs high precision and accuracy, so it also is equipped with high-precision encoder features to measure position and speed of the motor.

Further, another aspect relates to a depth position motor. For example, one presently disclosed exemplary embodiment of a depth position motor may be a linear motor to control depth of needle and device (out-of-plane, 3rd dimension). Similarly, such motor also needs high precision and accuracy, so it also is equipped with high-precision encoder features to measure position and speed of the motor.

A microcontroller (or set of microcontrollers, i.e. one or more processors) is preferably used as a control system of the overall device. Such an arrangement in some instances will be used in conjunction with circuitry to integrate the various mechanical components and sensors with a control algorithm. Such control algorithm will use the mechanical feedback system to accomplish various features.

For example, one such feature will be to automatically sense the temporal response of the heartbeat (dynamic “mechanical ECG” and “electrical ECG”) and map the local deformation activity of the heart in the region around injection. Such sensing mechanism will also be able to map tissue dysfunction to differentiate between healthy and non-healthy tissue and determine sites of payload injection.

Another feature accomplished through the control algorithm involving creation of a closed-loop system to automatically or semi-automatically guide injection using a combination of the syringe injection motor 110 and the depth position motor. Yet another feature accomplished through the control algorithm is to allow manual control of needle position using the rotational position motor and depth position motor, such as for multiple injection sites. A still further feature accomplished is to have the ability to place software safety limitations to ensure manual control and override. Another feature of the presently disclosed subject matter is to provide for achieving multiple, simultaneous injections in a defined local pattern using, for example, a multi-needle injection apparatus.

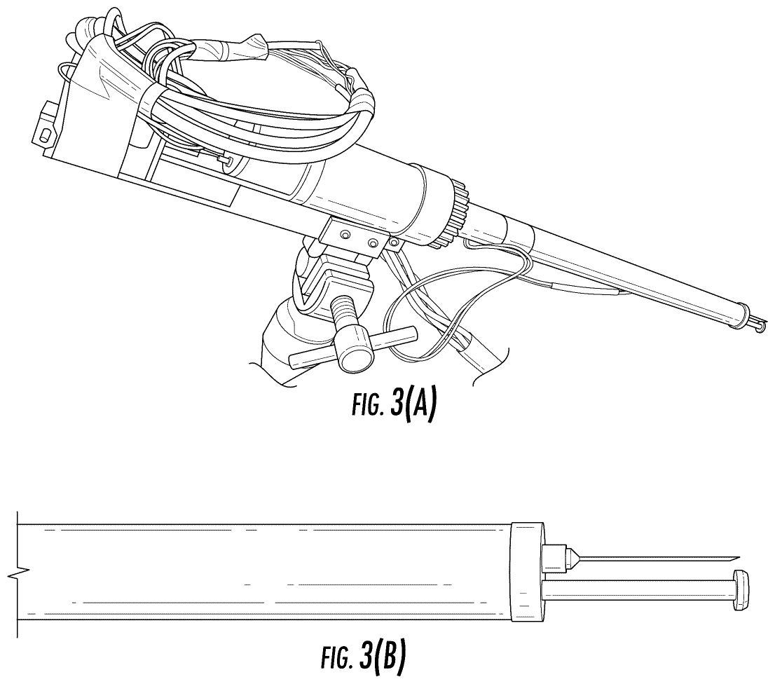

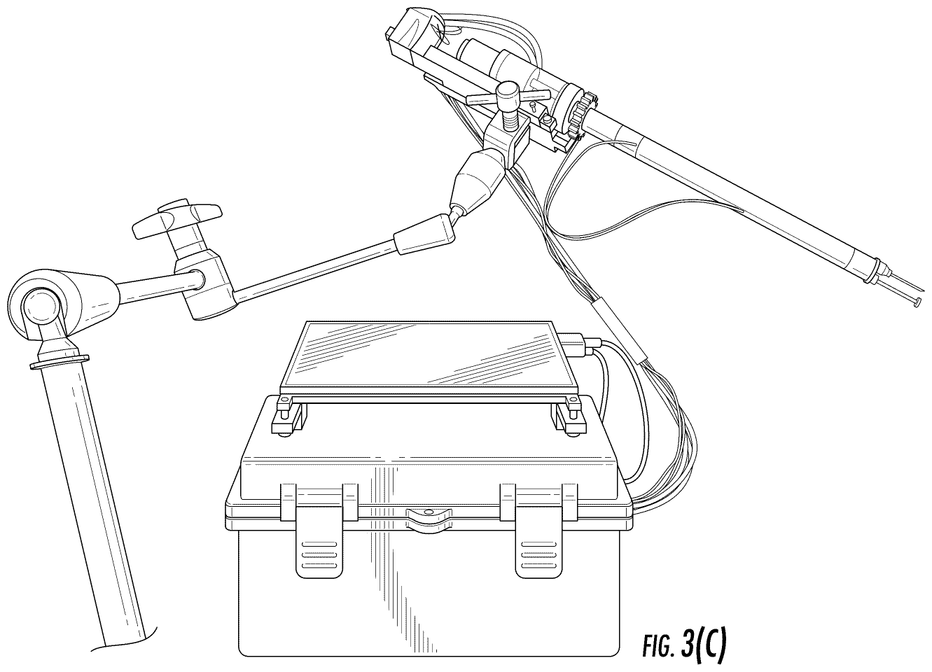

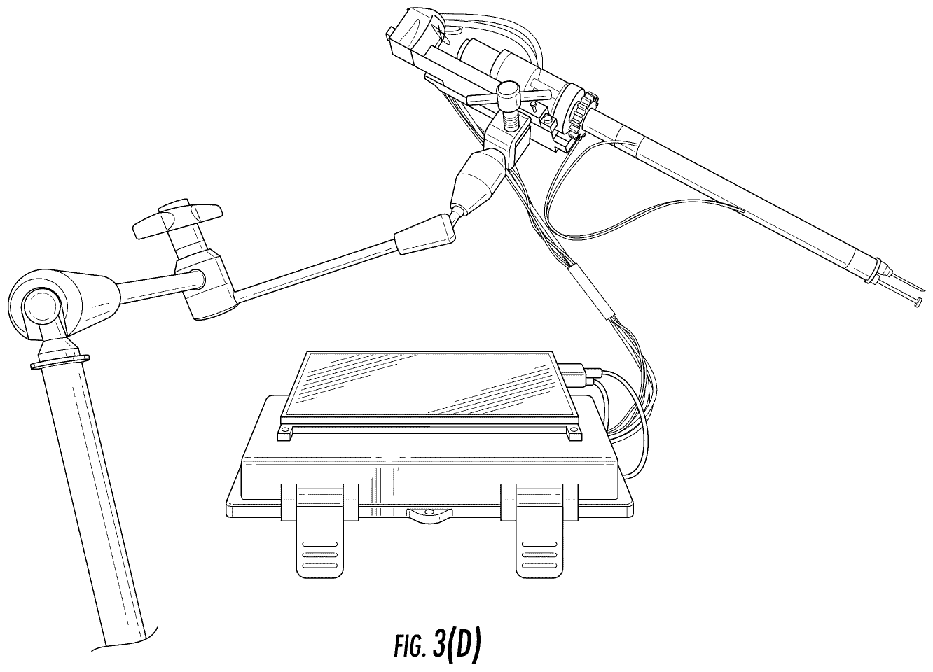

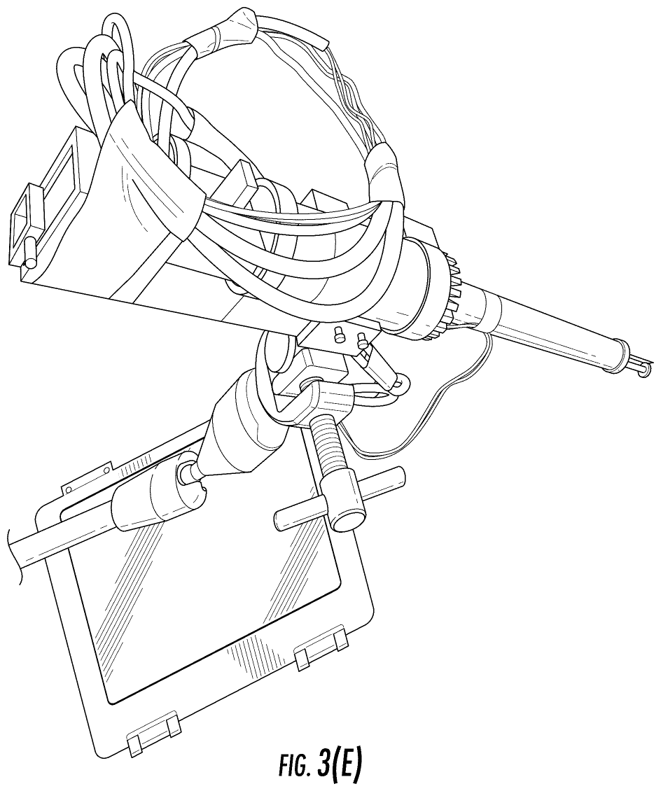

While FIGS. 2(A) and 2(B) illustrate 3D models of design schematics for the presently disclosed features as discussed above, FIGS. 3(A) through 3(E) illustrate images of produced (or manufactured) physical prototypes relating to the subject matter represented by FIGS. 2(A) and 2(B). In particular, FIG. 3(A) illustrates an isometric, partially side/partially top view of an exemplary embodiment of the subject matter represented by FIG. 2(A), mounted on a supporting surgical arm. FIG. 3(B) illustrates an enlarged view of a distal end of an exemplary embodiment of the subject matter represented by FIG. 2(A). In particular, close-up views are shown for needle 104 and physical sensor probe 106.

FIG. 3(C) illustrates an overall view of the subject matter of FIG. 3(A), and further illustrating connections with various supporting components of an exemplary embodiment of the overall device, including, for example, components supporting the presently disclosed electrical measurement system. FIG. 3(D) illustrates similar subject matter as that of FIG. 3(C) but from a different perspective. FIG. 3(E) illustrates an enlarged view of a portion of the FIG. 3(D) subject matter.

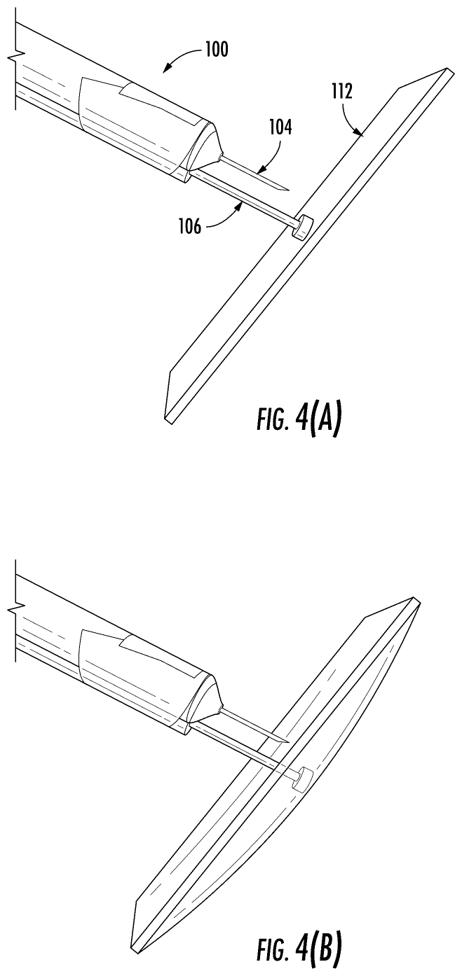

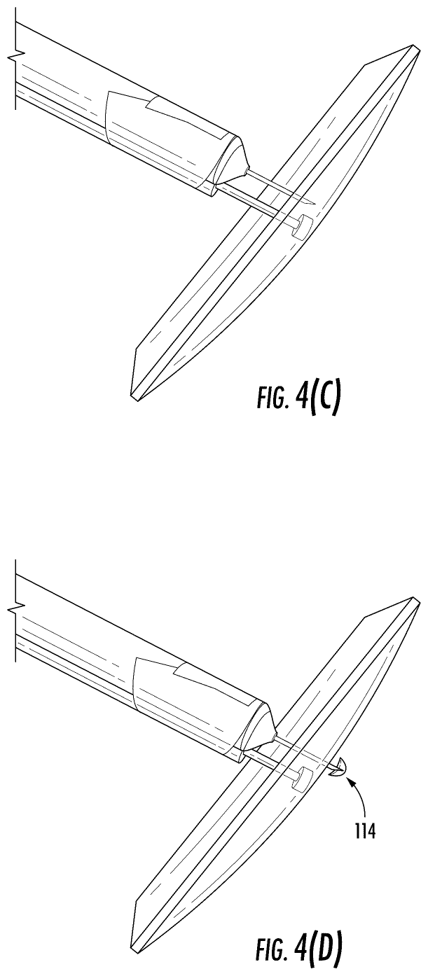

FIGS. 4(A) through 4(D) show successive images of an exemplary embodiment of an injection device or component of the presently disclosed subject matter engaging a plastic test material, in accordance with presently disclosed methodology. In each of the images of FIGS. 4(A) through 4(D), a section of flexible plastic test material generally 112 is held by hand in a position to be encountered by the presently disclosed injection device or component 100. The test material 112 is a stand-in for heart wall tissue, or other locations in or on a patient with which the presently disclosed subject matter might be used.

As shown by initial image FIG. 4(A) in the represented sequence, the physical sensor probe 106 has just begun to engage material 112 while needle 104 remains at a distance from the material 112. As shown by successive image FIG. 4(B), flexible material 112 is slightly moved by engagement of the physical sensor probe 106, just as could occur with actual patient tissue. Also, needle 104 is being advanced for penetrating material 112. Per further image FIG. 4(C) in this succession of images, physical sensor probe 106 continues to contact (and press against) material 112, while needle 104 is advanced for penetrating a distance into (and in this illustrative instance, actually through) material 112. In the final image of this represented sequence, FIG. 4(D) illustrates that while physical sensor probe remains in contact with force against material 112, and while needle 104 has penetrated material 112, the syringe is operated so that a dose 114 is administered from the needle 104.

Thus, the successive images of FIGS. 4(A) through 4(D) represent demonstration of a sequence in accordance with presently disclosed subject matter for injection of a drug (dose) through a representative plastic barrier.

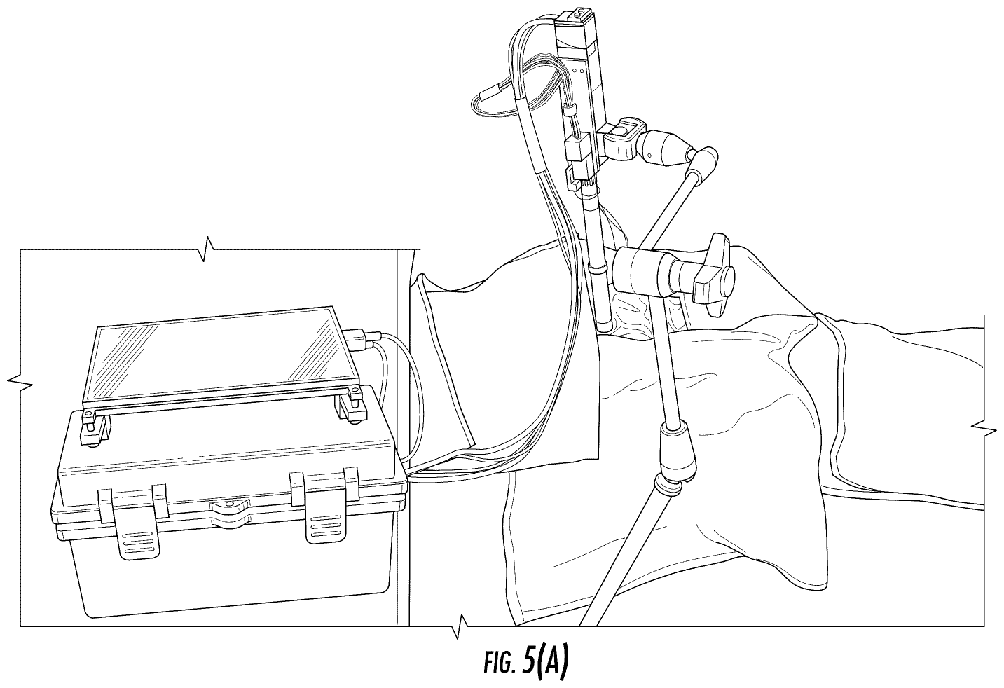

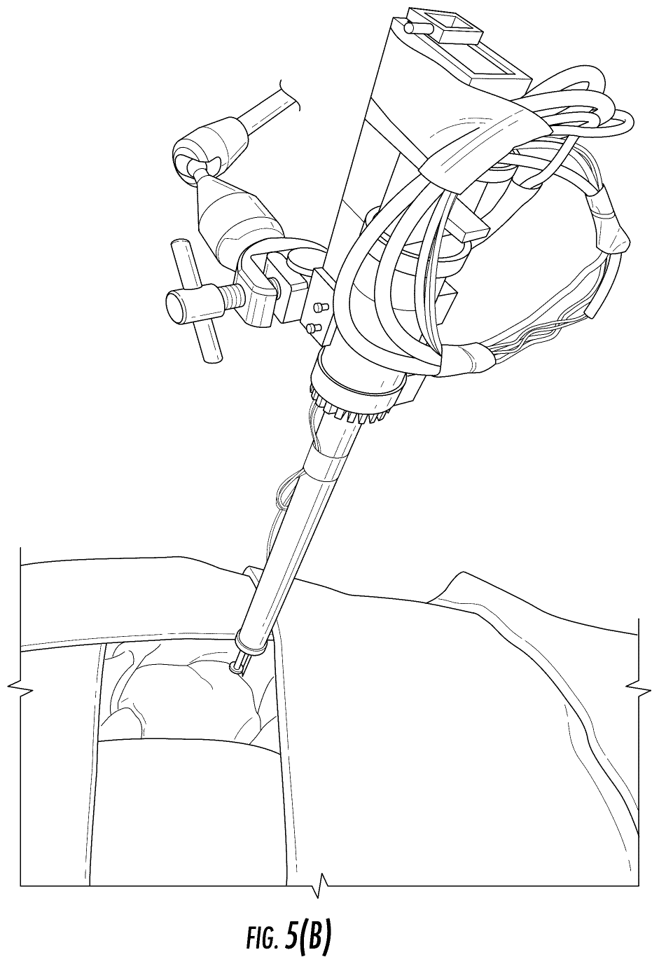

FIGS. 5(A) and 5(B) respectively illustrate images of a surgical set up of the presently disclosed subject matter during use, from opposite side perspectives relative to a subject during open-heart porcine (pig) surgery. As shown by both figures, but particularly by FIG. 5(B), the presently disclosed subject matter is supported by a surgical arm so that the distal end of the presently disclosed injection device is situated literally adjacent the exposed heart of the subject. Otherwise, as understood by those of ordinary skill in the art, the surgical arm is itself substantially laterally located relative to the subject, with the injection device positioned to be supported in a cantilevered position above and descending towards the subject's heart.

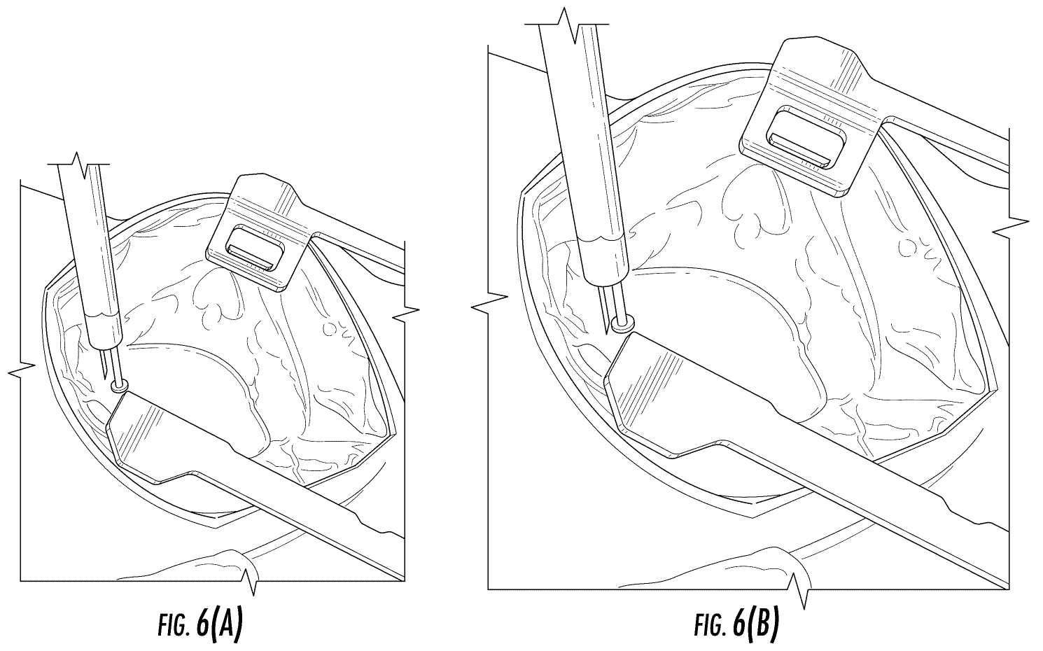

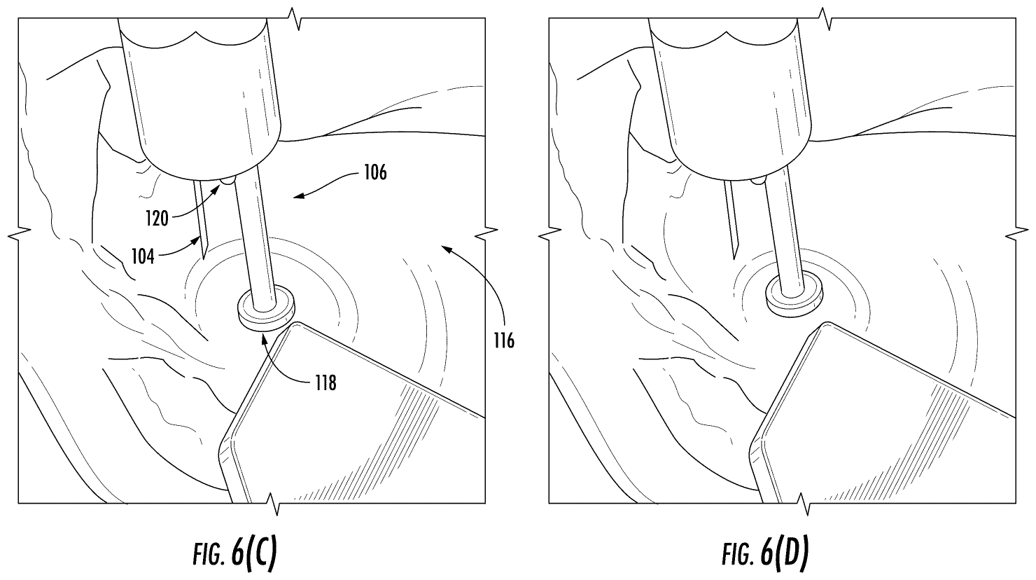

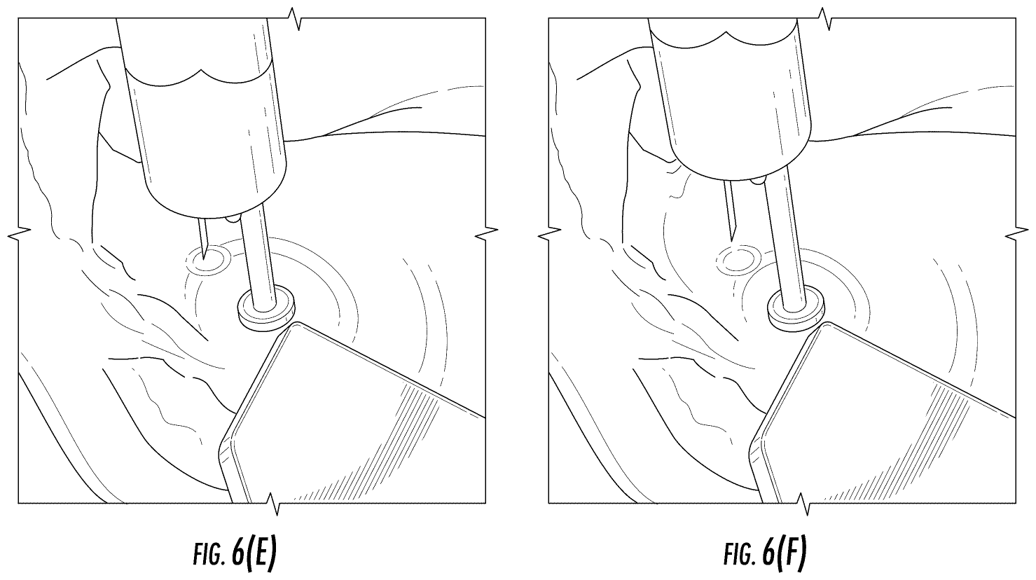

Similar to the successive images of FIGS. 4(A) through 4(D), FIGS. 6(A) through 6(E) represent demonstration of a sequence in accordance with presently disclosed subject matter for injection of a drug (dose) into a porcine subject heart rather than through a representative plastic barrier. Thus, FIGS. 6(A) through 6(E) show successive images of an exemplary embodiment of an injection device or component of the presently disclosed subject matter engaging a porcine subject heart, in accordance with presently disclosed methodology.

In each of FIGS. 6(A) through 6(E), physical sensor probe 106 is brought into, and kept in, mechanical contact with the subject beating heart 116. Therefore, as one of ordinary skill in the art will understand from the complete disclosure herewith, the far distal end 118 of the physical sensor probe 106 in effect “rides” against the beating heart. This means that the position of the probe distal end 118 relative to the end 120 of the device 100 is constantly changing as the heart 116 beats.

The progression of images per FIGS. 6(A) through 6(E) show that needle 104 is meanwhile brought closer to beating heart 116 until (FIG. 6(E)) it is quickly thrust into the heart and the syringe actuated for administration of a dose (drug) to the heart. FIG. 6(F) represents an exemplary retraction of needle 104 quickly after the dosage is administered. Thus, such FIGS. 6(A) through 6(E) represent in-vivo auto sensing and injection technology in accordance with presently disclosed subject matter.

It should be understood that the use of numerical reference characters throughout the figures and this specification are intended to refer to corresponding features in various figures, even if the numerical reference is not shown (for clarity) in each figure. For example, FIG. 6(C) includes a number of numerical references which correspond with various features throughout FIGS. 6(A) through 6(E) but without being labeled in every one of such figures. Similarly, for example, FIG. 4(A) includes labeled reference number features, to indicate corresponding features appearing in each of FIGS. 4(B) through 4(D), but not labeled in such other figures.

This written description uses examples to disclose the presently disclosed subject matter, including the best mode, and also to enable any person skilled in the art to practice the presently disclosed subject matter, including making and using any devices or systems and performing any incorporated methods. For example, while most of the description herein focuses on the heart, the presently disclosed technology could potentially be used for any accessible organ.

The patentable scope of the presently disclosed subject matter is defined by the claims, and may include other examples that occur to those skilled in the art. Such other examples are intended to be within the scope of the claims if they include structural and/or step elements that do not differ from the literal language of the claims, or if they include equivalent structural and/or elements with insubstantial differences from the literal languages of the claims. In any event, while certain embodiments of the disclosed subject matter have been described using specific terms, such description is for illustrative purposes only, and it is to be understood that changes and variations may be made without departing from the spirit or scope of the subject matter. Also, for purposes of the present disclosure, the terms “a” or “an” entity or object refers to one or more of such entity or object. Accordingly, the terms “a”, “an”, “one or more,” and “at least one” can be used interchangeably herein.

What is claimed is:

1. A method for semi-automated delivery of therapeutic payloads to a heart, comprising:
introducing into a patient's chest cavity an injection device supporting a needle and a movable physical sensor probe;
positioning the movable physical sensor probe into contact with the exposed heart of the patient;
tracking the beat pattern of the patient's heart by maintaining contact of the movable physical sensor probe with the patient's heart through its beat cycle; and
inserting the needle into the patient's heart and injecting a therapeutic payload via the needle.

2. The method according to claim 1, further comprising withdrawing the needle from the patient's heart.

3. The method according to claim 1, wherein the inserting, injecting, and withdrawing steps all occur during a selected portion of the beat cycle of the patient's heart.

4. The method according to claim 3, wherein the inserting and injecting steps are automatically timed to occur while the patient's heart is enlarged.

5. The method according to claim 1, further comprising determining a predetermined injection site on the heart of the patient.

6. The method according to claim 1, further comprising:
sensing linear motion of the movable physical sensor probe after it is brought into contact with the patient's heart; and
analyzing the sensed linear motion for tracking the beat pattern of the patient's heart.

7. The method according to claim 1, wherein the needle is 20 gauge or smaller, and is used for performing intra-myocardial injection into the patient's heart.

8. The method according to claim 1, further comprising using ECG measurements of the patient's heart in combination with physical tracking the patient's heart movable physical sensor probe to determine the beat pattern of the patient's heart.

9. The method according to claim 1, further comprising using a mounting component for supporting the injection device.

10. The method according to claim 1, further comprising using a payload syringe cartridge with the needle and having a capacity for delivering a total of 1 to 5 ml of a predetermined drug into the patient's heart.

11. The method according to claim 1, further comprising an operator manually controlling the position of the injection device for respective plural injection sites for a patient's heart.

12. The method according to claim 1, wherein introducing an injection device includes one of introducing an injection device through a patient's opened chest cavity and introducing an injection device into a patient's closed chest via minimally invasive port access.

13. The method according to claim 1, wherein the inserting and injecting includes making multiple, simultaneous injections in a defined local pattern.

14. The method according to claim 13, further comprising using a multi-needle injection apparatus for making the multiple, simultaneous injections in a defined local pattern.

15. A method for performing automated, targeted payload injection into the intra-myocardium of a patient, comprising:
controllably introducing a minimally invasive injection device into the patient's chest cavity; and
sensing the mechanical and electrical signals of the patient's heart and dynamically injecting a known quantity of therapeutic payload within a precise time window at a specific location, via a needle carried by the injection device.

16. The method according to claim 15, comprising using a closed feedback system of sensors and motors to semi-automatically carry out injections to the patient's beating heart.

17. The method according to claim 15, wherein introducing an injection device includes one of introducing an injection device through a patient's opened chest cavity and introducing an injection device into a patient's closed chest via minimally invasive port access.

18. The method according to claim 15, wherein the injecting includes making multiple, simultaneous injections in a defined local pattern using a multi-needle injection apparatus.

19. A system for semi-automated delivery of therapeutic payloads to a heart, comprising:
an injection device supporting a needle and a movable physical sensor probe;
a mounting component for supporting the injection device introduced into a patient's chest cavity, with the movable physical sensor probe into contact with the exposed heart of the patient;
a displacement sensor associated with the movable physical sensor probe, for measuring the amount of movement of the patient's heart while the movable physical sensor probe maintains contact with the patient's heart through its beat cycle;
an ECG device for providing electrical signals from the patient's heart;
a syringe associated with the needle for delivery of a predetermined drug to a patient's heart;
a syringe injection motor for controlling injection of the syringe;
a needle position motor for controlling the depth of the needle into a patient's heart; and
one or more processors programmed for tracking the beat pattern of the patient's heart by signals from the displacement sensor and the ECG device, and controlling the needle position motor and syringe injection motor for automatically inserting the needle into the patient's heart and injecting a therapeutic drug from the syringe via the needle.

20. The system according to claim 19, wherein the one or more processors are further programmed to automatically sense the temporal response of the heartbeat and map the deformation activity of the heart in the region around injection via the needle.

21. The system according to claim 19, further comprising:
a rotational position motor for in-plane control of a needle injection site and mechanical mapping of heart activity using the needle and movable physical sensor probe, respectively; and
wherein the one or more processors are further programmed to control the rotational position motor for selecting an injection site.

22. The system according to claim 21, wherein the one or more processors are further programmed to receive user input for manual selection of an injection site via control of the rotational position motor.

23. The system according to claim 19, wherein the one or more processors are further programmed for inserting the needle, injecting contents of the syringe, and withdrawing the needle from the patient's heart all occur during a selected portion of the beat cycle of the patient's heart.

24. The system according to claim 19, wherein the ECG device further includes embedded electrodes.

25. The system according to claim 19, wherein the needle comprises a multi-needle injection apparatus for making multiple, simultaneous injections in a defined local pattern.
